(19) United States
(12) Patent Application Publication
(10) Pub. No.: US 2022/0088150 A1
(43) Pub. Date: Mar. 24, 2022

(54) Octreotide Microsphere-Based Arterial Embolization for Treating Obesity

(71) Applicant: Biovena Science, LLC, New York, NY (US)

(72) Inventors: Fuad Nurili, New York, NY (US); Omer Aras, Edgewater, NJ (US)

(21) Appl. No.: 17/277,342

(22) PCT Filed: Sep. 19, 2019
(86) PCT No.: PCT/US19/51890

§ 371 Date: Mar. 18, 2021

Related U.S. Application Data
(60) Provisional application No. 62/733,704, filed on Sep. 20, 2018.

(51) Int. Cl. A61K 38/31; A61K 9/16; A61P 3/04; A61L 24/04

(52) U.S. Cl. CPC A61K 38/31; A61K 9/1647; A61P 3/04; A61L 2300/43; A61L 2430/36; A61L 2300/252; A61L 24/046

(57) ABSTRACT

This invention provides a method for causing weight loss in a subject comprising introducing biodegradable microspheres into one or more of the subject's gastric arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical octreotide; (iv) embolize gastric arterial vessels supplied by the one or more arteries into which they are introduced; and (v) release octreotide during embolization.

This application claims the benefit of U.S. Provisional Application No. 62/733,704, filed Sep. 20, 2018, the contents of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to methods for treating obesity via gastric arterial embolization. These methods employ octreotide-containing biodegradable microspheres.

### BACKGROUND OF HE INVENTION

Obesity

Obesity is a global epidemic. In an obese patient, excess body fat has accumulated to the extent that it may have a negative effect on health. Authorities view obesity as one of the most serious public health problems of the 21st century. Many treatment options exist for obesity.

The Stomach

The stomach is an endocrine organ that is critically involved in maintaining energy homeostasis, regulating satiety and body weight, and, to a substantial degree, regulating the cardiovascular system.

Ghrelin

Many satiety hormones have been discovered during the past decades. One of them is ghrelin, which has been shown to stimulate (i.e., prompt) food intake. In obese patients, eating fails to suppress ghrelin levels. This failure is believed to prevent feeling full after a meal and, thus, to cause overeating. Due to the strong hunger-inducing effect of ghrelin, this hormone has been a target for treating obesity and inducing weight loss. More recently, ghrelin has been shown to have a significant role in the long-term effects of weight loss in bariatric (obesity) surgery. Specifically, ghrelin levels in treated patients are much lower than those in untreated patients.

Microspheres

Biodegradable microspheres made of polylactic co-glycolic acid (PLGA) and polylactic acid (PLA) are known and suitable for transarterial drug delivery and transient embolization. PLGA is an FDA-approved biodegradable polymer. It has been extensively investigated in many medical and pharmaceutical fields due to its good biodegradability and biocompatibility. PLGA-containing microspheres have shown sustained release characteristics due to degradation and diffusion mechanisms. Biodegradibility advantages include potential reduction in the occurrence of post-embolization syndrome tissue inflammation and fibrosis, risks arising from non-target embolization, and the possibility of repeated interventions after vessel recanalization.

Embolization

Embolization studies have been performed in animals and humans. The following are relevant examples.

Arepally, et al, (2007) studied catheter-directed gastric artery chemical embolization for modulation of systemic ghrelin levels in a porcine model. The study showed a reduction in systemic levels of ghrelin without significant change in weight. Arepally, et al. (2008) dealt with the same subject matter. The investigators evaluated the natural weight gain in young swine after gastric artery chemical embolization or a sham procedure was performed. A significant reduction in weight gain in the treated group (7.8% at week 4), compared with the control group (15.1% at week 4), was found (p<0,04). Paxton, et al. studied bariatric embolization for suppression of ghrelin in a porcine model. This study showed a 55% reduction in serum levels of ghrelin after using 40 μm calibrated Embozene™ microspheres (CeloNova). In addition, the experimental group had less weight gain during the eight weeks after the procedure than did the control group that underwent a sham procedure (3.6 vs 9.4 kg, respectively; p=0.025). Bawudun, et al. studied ghrelin suppression and fat loss after left gastric artery embolization (LGAE) in a canine model. This study showed decreased plasma levels of ghrelin, body weight, and subcutaneous fat in dogs that underwent LGAE, compared with control dogs that received a sham treatment.

Gunn and Oklu studied weight loss following left gastric artery embolization in humans. They compared 19 patients who underwent LGAE with 28 patients who underwent embolization of a separate artery for gastrointestinal bleeding. The study found a statistically significant reduction in the body weight of patients in the LGAE group (7.3%) versus the control group (2%) in the first three months after the procedure was performed. No statistically significant difference was seen at later points in time, which were not standardized (13.6 months in the experimental group vs four months in the control group). In addition, both groups included patients with documented malignancies.

Kipshidze, et al. studied left gastric artery embolization for weight loss in humans. This study used a prospective, single-arm human model in which five patients received LGAE, mean (±SD) value. The subjects' weight decreased from 128.12±24 kg to 114.86±21 kg, and BMI decreased from 42.26±6.8 to 37.86±5.7. There was no control group. Three out of five patients complained of epigastric pain during the first few hours after the procedure, but esophago-gastroduodenoscopy showed no significant complications.

Finally, Weiss, et al. studied the clinical safety of bariatric arterial embolization. In this study, the left gastric artery, with or without the gastroepiploic artery, was embolized in five patients with a technical success rate of 100%. There were no major adverse effects. There were two minor adverse effects—subclinical pancreatitis and a mucosal ulcer that had healed by the time of three-month endoscopy. A hospital stay of less than 48 hours for routine supportive care was provided for three patients. A mean excess weight loss of 5.9%−/+2.4 and 9.0%−/+4.1 was noted at one month and at three months, respectively. The mean change in serum ghrelin was 8.7%−/+34.7 and 217.5%−/+29 at one month and three months, respectively. Mean changes in serum glucagon-like peptide 1 and peptide YY were 106.6%−/+208.5 and 17.8%−/++54.8 at one month. There was a trend toward improvement in QOL parameters. Hunger/appetite scores decreased in the first two weeks after the procedure and then rose without reaching pre-procedure levels.

Octreotide

Octreotide is an eight-amino acid synthetic analog of somatostatin that resembles the native polypeptide in its activity in suppressing levels and activity of growth hormone, insulin, glucagon and many other gastrointestinal peptides. It is also used for the treatment of growth hormone-producing tumors. Moreover, it is known that continuous infusion of octreotide into the gastric artery of the vascularly perfused rat stomach suppresses ghrelin secretion in both a dose- and time-dependent manner. Octreotide has an elimination half-life of 1.5 hours. It reduces splanchnic blood flow in healthy human subjects and delays gastric emptying. Furthermore, octreotide has been used experimentally in attempts to treat obesity, particularly obesity caused by lesions in the hunger and satiety centers of the hypothalamus (a region of the brain central to the intravenous regulation of food intake and energy expenditure).

Previous Studies

Lustig et al. [1] used somatostatin analogues to inhibit insulin hypersecretion. In the first pilot study, eight children with intractable, hypothalamic obesity (BMI 36±2.5 kg/m²) received octreotide for six months at a dosage of 5-15 μg/kg/day, in three divided doses. In comparison with a six-month pre-study observation period, patients exhibited greater weight loss (p=0.04) and BMI decrease (p=0.0001) after treatment. The degree of weight loss correlated both with changes in insulin response on oral glucose tolerance test (OGTT) and changes in leptin levels. Calorie intake decreased by approximately 700 kcal/day and correlated with weight loss. [1]

The same group performed a second study. That study involved a double-blind placebo-controlled, six-month trial of octreotide in 18 subjects (age 13.8±1.2 years, BMI 36.4±2.4 kg/m²) with hypothalamic obesity. [2] Patients received octreotide subcutaneously (5-15 μg/kg/day) or placebo for six months. Octreotide was effective in stabilizing weight and BMI in the active-treatment group compared with placebo, but weight loss was less pronounced than in the previous study.

Velasquez-Mieyer et al. performed an uncontrolled study of long-acting-release octreotide (octreotide LAR) on an adult population. Forty mg of drug was administered to 44 severely obese adults (BMI 44.3±1.0 kg/m²) for six months. The results showed that significant insulin suppression was achieved with octreotide LAR. This effect paralleled improvements in insulin sensitivity and BMI.

A trial by Bai, et al., showed that average body weight loss after trans-catheter LGAE for treating five obese patients at three, six, and nine months was 8.28±7.3 kg (p=0.074), 10.42±8.21 kg (p=0.047), and 12.9±14.66 kg (p=0.121), respectively. The level of serum ghrelin decreased by 40.83% (p=0.009), 31.94% (p=0.107), and 24.82% (p=0.151) at three, six, and nine months after LGAE, respectively. The nine-month data from this study on five patients showed a trend toward weight loss, which was 8.28±7.3 kg (p=0.074), 10.42±8.21 kg (p=0.047), and 12.9±14.66 kg (p=0.121) at three, six, and nine months, respectively. Compared with previous short-term clinical studies, these results showed persistent weight loss even at nine months after LGAE.

Finally, in the SCALE trial, 3 mg of liraglutide induced relative weight loss of 5% in more than 50% of patients and 10% in more than 25% of patients in a large and well-characterized cohort after one year (Davies, et al.). In addition, six months after MedSil® balloon insertion, overall weight loss was 18.4 kg in their patient cohort (Buzga, et al.),

An Unmet Need

There is an unmet need for a superior way to employ gastric embolization to induce weight loss in obese patients.

### SUMMARY OF THE INVENTION

This invention provides a method for causing weight loss in a subject comprising introducing biodegradable microspheres into one or more of the subject's gastric arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical octreotide; (iv) embolize gastric arterial vessels supplied by the one or more arteries into which they are introduced; and (v) release octreotide during embolization.

This invention also provides a method for causing weight loss in a human subject having a BMI of 40 or higher comprising introducing biodegradable microspheres into the subject's left gastric artery, wherein the microspheres (i) have a d₉₀ value from 70 μm to 150 μm; (ii) comprise PLA and PLGA at a PLA:PLGA molar ratio of 45:55; (iii) carry from 10 mg to 20 mg of pharmaceutical octreotide; (iv) embolize gastric arterial vessels supplied by the left gastric artery; and (v) release octreotide during embolization.

### BRIEF DESCRIPTION OF HE FIGURES

FIG. 1 This figure shows a scheme depicting microparticle synthesis via a double emulsion method.

FIG. 2 This figure shows the size distribution of microparticles 10%<73 μm; 50%<181 μm; and 90%<414 μm.

FIG. 3 This figure shows SEM images of manufactured microparticles.

FIGS. 4A and 4B FIG. 4A shows the targeted area (arrow) for gastric embolization. FIG. 4B shows a photomicrograph of a histologic section of fundus showing atrophy of the residual gland tissue, but well-preserved mucosa.

FIGS. 5A and 5B These figures show immunohistochemical staining of the gastric fundus for ghrelin-expressing cells. FIG. 5A shows a control group displaying multiple dark foci representing ghrelin-positivity. In FIG. 5B, treatment with octreotide-loaded microspheres shows a greatly decreased number of ghrelin-expressing cells due to ischemic and octreotide's inhibitory effects.

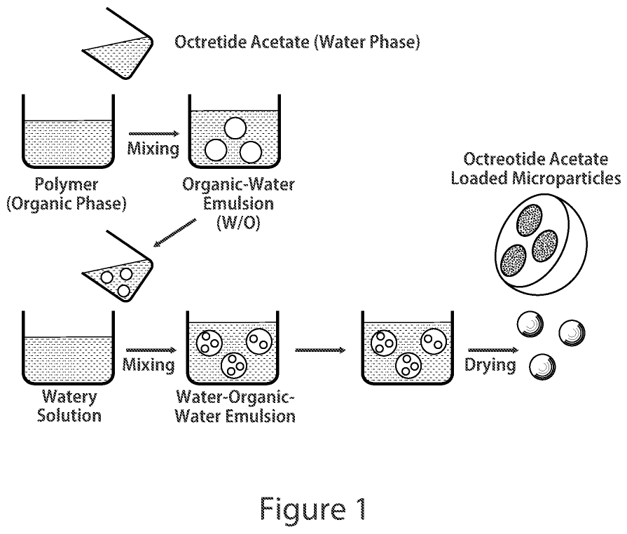

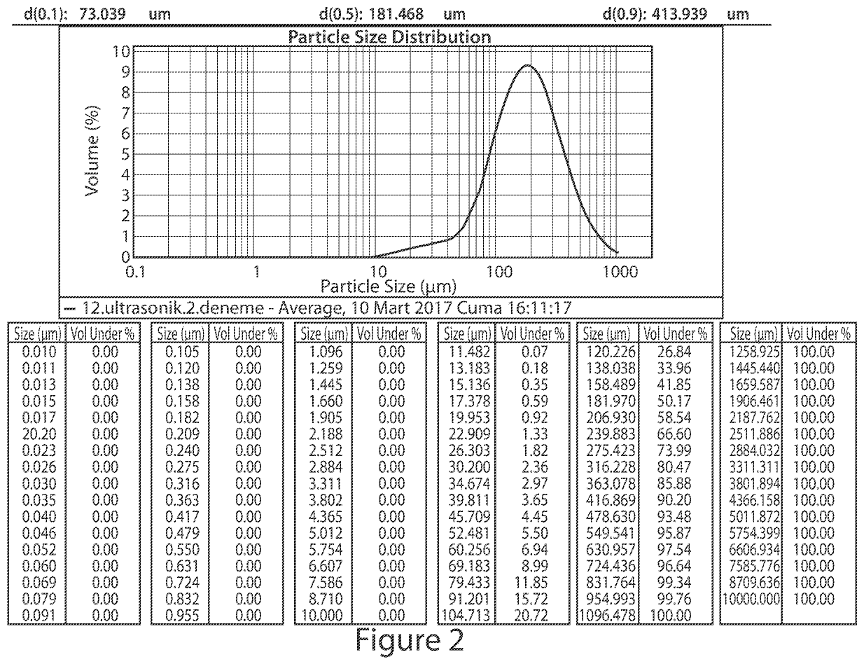

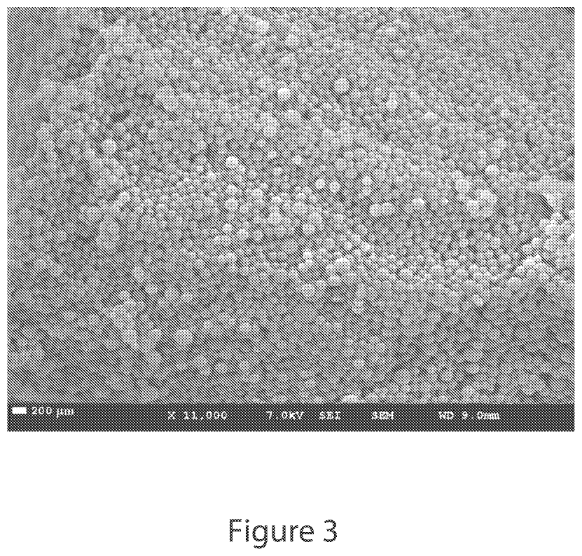

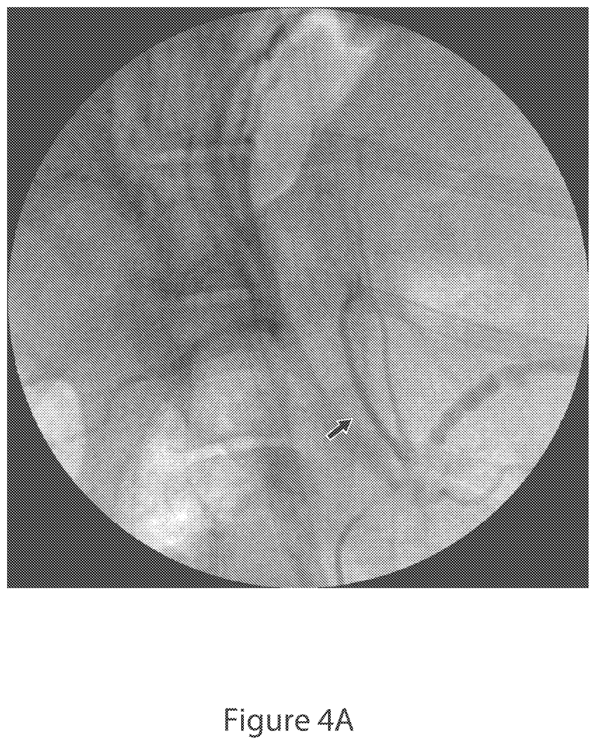

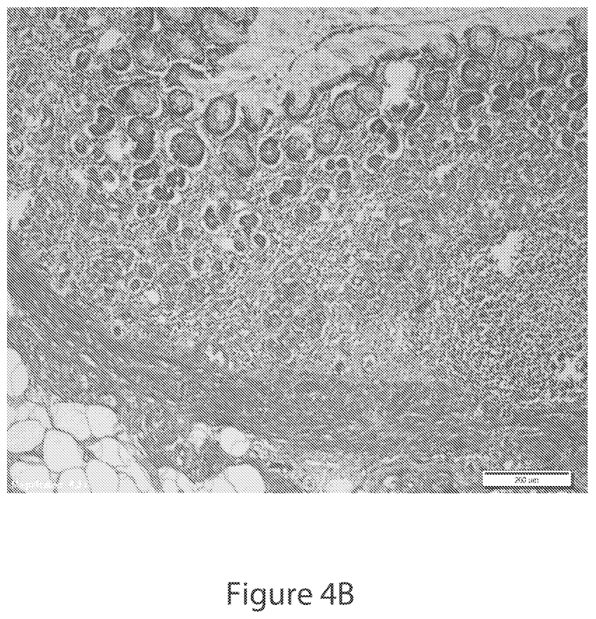

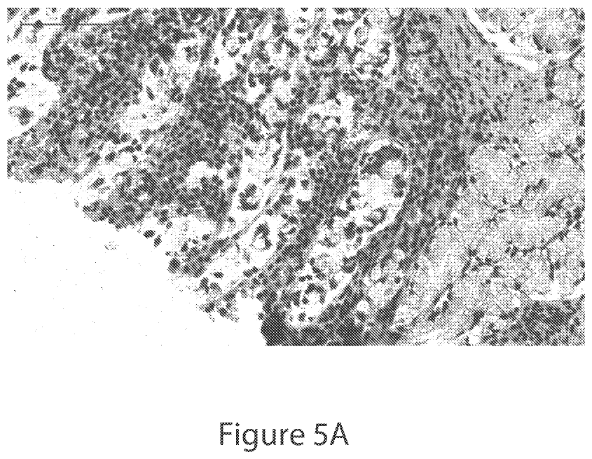

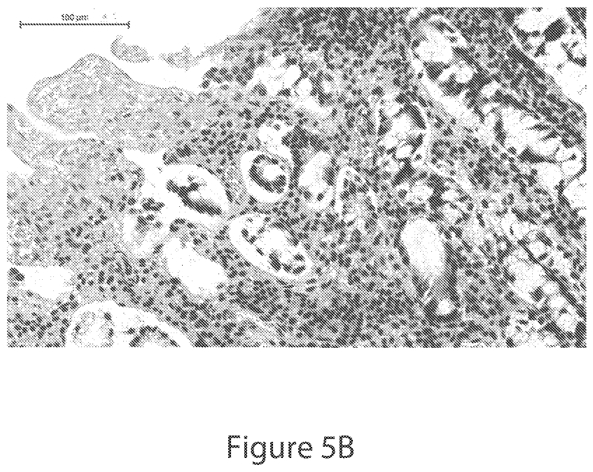

### DETAILED DESCRIPTION OF THE INVENTION

This invention provides new methods for causing weight loss in obese patients. These methods employ octreotide-containing biodegradable microspheres to embolize patients' gastric arteries.

### Definitions

In this application, certain terms are used which shall have the meanings set forth as follows.

As used herein, the term “arterial vessel” means an artery, an arteriole or a capillary.

As used herein, the term “biodegradable microsphere” means a polymeric sphere that (i) has a diameter from 40 μm to 500 μm, (ii) can non-covalently carry a therapeutic agent (e.g., octreotide acetate), and (iii) depending on its polymeric composition, degrades over a period lasting from days to months when placed in the human circulatory system. A biodegradable microsphere can be, for example, a sphere comprising PLA and PLGA (e.g., having a PLA:PLGA molar ratio of 45:55) that (i) has a diameter of 100 μm, (ii) can non-covalently contain octreotide acetate, and (iii) degrades over a period lasting two months when embolizing a gastric arterial vessel. Biodegradable PLA/PLGA microspheres are commercially available from, among other sources, Millipore-Sigma in the form of Degrader® products (Burlington, Mass.).

As used herein, a biodegradable microsphere comprising PLA “and/or” PLGA can be a biodegradable microsphere made solely of PLA, made solely of PLGA, or made of a combination of FLA and PLGA. The higher a microsphere's PLA content, the slower it degrades and, thus, the more stable it is. Conversely, the higher a microsphere's PLGA content, the faster it degrades and the less stable it is. In one embodiment, the biodegradable microsphere is made of a combination of PLA and PLGA wherein the molar ratio of PLA to PLGA is 5:95, 10:90, 15:85, 20:80, 25:75, 30:70, 35:65, 40:60, 45:55, 50:50, 55:45, 60:40, 65:35, 70:30, 75:25, 80:20, 85:15, 90:10, or 95:5. In another embodiment, the biodegradable microsphere is made of a combination of PLA and PLGA wherein the molar ratio of PLA to PLGA is from 5:95 to 20:80, from 20:80 to 40:60, from 40:60 to 50:50, from 40:60 to 60:40, from 50:50 to 60:40, from 60:40 to 80:20, or from 80:20 to 95:5. The population of biodegradable microspheres used in this invention can be homogeneous or heterogeneous with respect to the microspheres' molar ratio of PLA to PLGA. In one embodiment, the population of biodegradable microspheres is homogeneous with respect to the microspheres' molar ratio of PLA to PLGA (e.g., the population includes only microspheres wherein the molar ratio of PLA to PLGA is 45:55). In another embodiment, the population of biodegradable microspheres is heterogeneous (e.g., the population includes both (i) microspheres wherein the molar ratio of PLA to PLGA is 45:55, and (ii) microspheres wherein the molar ratio of PLA to PLGA is 50:50). Methods of preparing biodegradable microspheres using PLA and PLGA are known, as are methods of preparing homogeneous and heterogeneous populations thereof having defined molar ratios of PLA to PLGA. Such calibrated biodegradable microspheres comprising PLA and PLGA are commercially available from, among other sources, Millipore-Sigma in the form of Degradex® products (Burlington, Mass.).

As used herein, the term “carry”, with respect to pharmaceutical octreotide and a biodegradable microsphere, means that the pharmaceutical octreotide is non-covalently bound to, or otherwise contained in or on, the biodegradable microsphere in a manner permitting release from the microsphere during its biodegradation.

As used herein, the phrase “causing weight loss” in a subject includes, without limitation, (i) causing a loss of at least 10%, at least 15%, at least 20%, at least 25%, at least 30%, at least 35%, at least 40%, at least 50%, at least 55%, or at least 60% of the subject's body weight; (ii) causing a loss of from 10% to 15%, from 15% to 20%, from 20% to 25%, from 25% to 30%, from 30% to 35%, from 35% to 40%, from 40% to 50%, or from 50% to 60% of the subject's body weight; and (iii) causing a loss of 10%, 15%, 20%, 25%, 30%, 35%, 40%, 50%, 55% or 60% of the subject's body weight. Preferably, weight loss as exemplified above is measured over a time period of from one to six months (e.g., after one month, after two months, after three months, after four months, after five months, or after six months). In a further embodiment of this invention, the subject method, rather than causing weight loss, either stops weight loss or reduces its rate (e.g., by limiting weight gain over the above time period to below 1%, 2%, 3%, 4% or 5% of the subject's body weight).

As used herein, the term “d₉₀ value”, with respect to a population of biodegradable microspheres having a specified size range, means that 90% of the biodegradable microspheres have a diameter in the specified range.

As used herein, the term “embolize”, with respect to an arterial vessel, means to occlude blood flow through the vessel by remaining in place within it. In one embodiment of this invention, embolize means to occlude blood flow through the vessel by at least 70%, at least 75%, at least 80%, at least 85%, at least 90%, or at least 95%.

As used herein, “introducing”, with respect to biodegradable microspheres, means delivering to a specified part of the body, such as a gastric artery. Methods of introducing biodegradable microspheres to an artery to embolize that artery or arterial vessels supplied by it are known. See, e.g., Sommer, et al., Liu, et al., Sheth, et al., van Elk, et al., Yan, et al., and Weng, et al.

As used herein, the term “octreotide” shall mean D-phenylalanyl-L-cysteinyl-L-phenylalanyl-D-tryptophyl-L-lysyl-L-threonyl-L-cysteinyl-L-threoninol (2→7)-disulfide (IUPAC name). Octreotide is a somatostatin analogue, and is used to treat acromegaly and complications associated with certain types of tumors.

As used herein, the term “pharmaceutical octreotide” includes, without limitation, octreotide and pharmaceutical salts and esters thereof. Pharmaceutical octreotide includes, for example, octreotide acetate. Octreotide acetate is commercially available, and is sold by Novartis under the trade name Sandostatin®.

As used herein, the term “subject” includes, without limitation, a mammal such as a human, a non-human primate, a dog, a cat, a horse, a sheep, a goat, a cow, a rabbit, a pig, a rat and a mouse. Preferably, the subject is human. In one embodiment, the human subject has a body mass index (BMI) of 30 or higher, 35 or higher, 40 or higher, 45 or higher, 50 or higher, 55 or higher, 60 or higher, 65 or higher, or 70 or higher. In another embodiment, the human subject has a BMI from 30 to 35, from 30 to 40, from 30 to 45, from 30 to 50, from 30 to 55, from 30 to 60, from 30 to 65, from 30 to 70, from 40 to 45, from 40 to 50, from 40 to 55, from 40 to 60, from 40 to 65, from 40 to 70, from 45 to 50, from 45 to 55, from 45 to 60, from 45 to 65, from 45 to 70, from 50 to 55, from 50 to 60, from 50 to 65, from 50 to 70, from 55 to 60, from 55 to 65, from 55 to 70, from 60 to 65, from 60 to 70, or from 65 to 70.

As used herein, the term “therapeutically effective amount”, with respect to pharmaceutical octreotide and biodegradable microspheres, refers to the amount of pharmaceutical octreotide collectively carried by the total dose of biodegradable microspheres introduced into the subject's one or more gastric arteries. In one embodiment, the effective amount is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg. In another embodiment, the effective amount is from 5 mg to 10 mg, from 10 mg to 15 mg, from 15 mg to 20 mg, from 20 mg to 25 mg, from 25 mg to 30 mg, from 30 mg to 35 mg, from 35 mg to 40 mg, from 40 mg to 45 mg, from 45 mg to 50 mg, from 50 mg to 55 mg, from 55 mg to 60 mg, from 60 mg to 65 mg, from 65 mg to 70 mg, from 70 mg to 75 mg, from 75 mg to 80 mg, from 80 mg to 85 mg, from 85 mg to 90 mg, from 90 mg to 95 mg, or from 95 mg to 100 mg. In a further embodiment, the effective amount is from 1 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, or from 90 mg to 100 mg. In yet a further embodiment, the effective amount is from 1 mg to 20 mg, from 20 mg to 40 mg, from 40 mg to 60 mg, from 60 mg to 80 mg, or from 80 mg to 100 mg.

### Embodiments of the Invention

This invention solves an unmet need in the art by providing an unexpectedly superior way to cause weight loss in an obese subject. The invention does this via embolizing the subject's gastric arteries with octreotide-carrying microspheres that release octreotide over time.

Specifically, this invention provides a method for causing weight loss in a subject comprising introducing biodegradable microspheres into one or more of the subject's gastric arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical octreotide; (iv) embolize gastric arterial vessels supplied by (i.e., distal to) the one or more arteries into which they are introduced; and (v) release octreotide during embolization. Preferably, the subject is human.

In the instant method, the biodegradable microspheres can be administered via one or more suitable gastric arteries. Preferably, the subject method comprises introducing the biodegradable microspheres into the subject's left gastric artery. Methods of arterial microsphere delivery generally are known generally, as are methods of gastric arterial microsphere delivery.

The human subject treated in the instant method can have any BMI above normal, such as a BMI of 30 or higher, 40 or higher, or 50 or higher. Preferably, the subject has a BMI of 40 or higher.

In one embodiment of the instant method, each biodegradable microsphere comprises PLA and PLGA. The PLA:PLGA molar ratio of these microspheres can be set at whatever ratio is desirable regarding stability and duration period of drug delivery. In one embodiment, each microsphere comprises PLA and PLGA at a PLA:PLGA molar ratio from 40:60 to 50:50. Preferably, the PLA:PLGA molar ratio is 45:55.

The biodegradable microspheres of the instant method can have any d₉₀ value permitting gastric embolization. Such d₉₀ values include, without limitation, 50 μm, 100 μm, 150 μm, 200 μm, 250 μm and 300 μm. In one embodiment of the instant method, the biodegradable microspheres have a d₉₀ value from 50 μm to 100 μm, from 100 μm to 150 μm, from 150 μm to 200 μm, from 200 μm to 250 μm, or from 250 μm to 300 μm. In another embodiment, the biodegradable microspheres have a d₉₀ value from 50 μm to 200 μm. In a further embodiment, the biodegradable microspheres have a d₉₀ value of 70 μm, 80 μm, 90 μm, 110 μm, 120 μm, 130 μm or 140 μm. Preferably, the biodegradable microspheres have a d₉₀ value from 70 μm to 150 μm.

In one embodiment, the therapeutically effective amount of pharmaceutical octreotide is from 5 mg to 50 mg. In another embodiment, it is from 10 mg to 20 mg (e.g., 15 mg). In a further embodiment, it is from 20 mg to 30 mg.

The instant method employs biodegradable microsphere-induced embolization to permit the extended and localized release of octreotide. Ideally, the duration of embolization lasts one or more weeks (e.g., one week, two weeks, three weeks, four weeks, five weeks, six weeks, seven weeks, eight weeks, nine weeks, 10 weeks, 11 weeks or 12 weeks). Preferably, the embolization lasts from four to eight weeks. The duration of embolization can be adjusted by adjusting the PLA:PLGA molar ratios of the biodegradable microspheres. In one embodiment, the instant method is performed only once for a given subject. In another embodiment, the instant method is performed a plurality of times (e.g., two times, three times, four times, five times or more). In that embodiment, each subsequent time the method is performed, it is performed after a suitable period has lapsed since the preceding time the method was performed. This suitable time can be, for example, one month, two months, three months, four months, five months, six months, one year, or longer.

This invention further provides a method for causing weight loss in a human subject having a BMI of 40 or higher comprising introducing biodegradable microspheres into the subject's left gastric artery, wherein the microspheres (i) have a d₉₀ value from 70 μm to 150 μm; (ii) comprise PLA and PLGA at a PLA:PLGA molar ratio of 45:55; (iii) carry from 10 mg to 20 mg of pharmaceutical octreotide; (iv) embolize gastric arterial vessels supplied by the left gastric artery; and (v) release octreotide during embolization. Where applicable, the embodiments described above for the first instant method are also envisioned for this method.

This invention still further provides an article of manufacture for use in causing weight loss in a subject by introducing biodegradable microspheres into one or more of the subject's gastric arteries. The article comprises (a) biodegradable microspheres, wherein the microspheres (i) have a d₉₀ value from 40 μm to 500 μm, (ii) comprise polylactic acid (PLA) and/or polylactic co-glycolic acid (PLGA), (iii) carry a therapeutically effective amount of pharmaceutical octreotide, (iv) embolize gastric arterial vessels supplied by the one or more gastric arteries into which they are introduced, and (v) release octreotide during embolization; and (b) a label instructing the user to introduce the biodegradable microspheres into one or more of a subject's gastric arteries so as to cause weight loss in the subject. Where applicable, the embodiments described above for the instant methods are also envisioned for this article of manufacture.

Finally, this invention provides an article of manufacture for use in causing weight loss in a human subject having a BMI of 40 or higher by introducing biodegradable microspheres into the subject's left gastric artery. The article comprises (a) biodegradable microspheres; wherein the microspheres (i) have a d₉₀ value from 70 μm to 150 μm, (ii) comprise FLA and PLGA at a PLA:PLGA molar ratio of 45:55, (iii) carry from 10 mg to 20 mg of pharmaceutical octreotide, (iv) embolize gastric arterial vessels supplied by the left gastric artery, and (v) release octreotide during embolization; and (b) a label instructing the user to introduce the biodegradable microspheres into a subject's left gastric artery so as to cause weight loss in the subject, wherein the subject is a human having a BMI of 40 or higher. Where applicable; the embodiments described above for the instant methods are also envisioned for this article of manufacture.

Wherever applicable, the methods of the subject invention may also be performed using an octreotide analog, a somatostatin analog or a ghrelin inhibitor. These agents include, without limitation, somatostatin, MK678, WOK4D, prosomatostatin, somatostatin-28, somatostatin-14, lanreotide, seglitide, vapreotide, AN-238, SMS 201-995, SDZ CO 611, RC-160, SMS-D70, SOM 230, KE 108, CGP 23996, BIM 23014, L362.855, L054.522, and a ghrelin o-acyltransferase (GOAT) inhibitor. The dose of each of these agents can be, for example, the dose recommended for its individual administration or the dose set forth above for octreotide in the subject invention, as appropriate. Therefore, the various embodiments of the invention relating to octreotide-based methods also apply, mutatis mutandis, to these agents.

This invention will be better understood by reference to the examples which follow, but those skilled in the art will readily appreciate that the specific examples detailed are only illustrative of the invention as described more fully in the claims which follow thereafter.

### EXAMPLES

### Example 1—Porcine Study

Twelve large pigs were randomly assigned to the embolization group (n=6) or control group (n=6). Selective angiography was performed in all animals after general anesthesia was induced. In the embolization group, octreotide-loaded PLGA/PLA microspheres (60-150 μm) were used to embolize the gastric arteries. Weight and fasting plasma ghrelin levels were compared in swine at baseline and at weeks 1-5. Swine in the embolization group showed a significant decrease in serum ghrelin values. In the embolization group, tremendous weight loss was seen in the first three weeks along with decreased appetite. Angiographies in the embolization group prior to necropsy showed total revascularization of gastric arteries. Histochemical staining showed preservation of overall tissue architecture and parietal cells. Immunochemical examination showed significant reduction in ghrelin-expressing cells in all stomach sections.

### Example 2—Human Study

The purpose of this study is to (i) assess the safety profile of gastric artery embolization with octreotide-loaded PLGAIPLA microspheres, (ii) confirm sustained post-procedural weight loss, and (iii) pathologically assess post-procedural metabolic effects.

Study Design

The primary purpose of the study is treatment. The study's title is “Embolization of Gastric Arteries with Octreotide loaded PLGA/PLA biodegradable microspheres Trial for the Obese Patients.”

Intervention Details

Procedure: Octreotide-loaded PLGA/PLA microspheres will be used intra-arterially to occlude the gastric artery and its branches. There will be a 30-day post-embolization endoscopic biopsy.

Outcome Measures

Primary outcome measures include adverse events [time frame=5 years]. Safety outcomes involve the use of gastric artery embolization.

Secondary outcome measures include the following: (i) Change in body mass index (BMI, kg/m²) [time frame=5 years]. (ii) Percentage estimated body weight loss (EBWL) [time frame=5 years]. (iii) Change in concentration of ghrelin-producing cells [time frame=30 days post-operative].

Concentration of ghrelin-producing cells pre-embolization obtained through endoscopic biopsy and measured by cells per milliliter will be subtracted from the concentration of ghrelin-producing cells obtained post-embolization from endoscopic biopsy. This value will be converted into a percentage change and reported.

Eligibility

Subjects 18 years and older, of all sexes, are eligible. Healthy volunteers are accepted.

Criteria

Inclusion criteria include the following: (1) Patients who are bariatric surgical candidates; however, have refused surgery. (2) BMI>40. (3) BMI between 35 and 40 with medical comorbidities. (4) Patients who meet criteria for medical management of obesity with BMI≥35. (5) Age≥18 years. (6) Willing, able and mentally competent to provide written informed consent.

Exclusion criteria include the following: (1) Age less than 18 years of age. (2) Inability to lay supine on an angiographic table >500 lbs due to table weight limits. (3) Inappropriate anesthesia risk as determined by certified anesthesia provider. (4) Presence of a contraindication to endovascular therapy. (5) Major surgery within the past eight weeks. (6) Previous gastric, pancreatic, hepatic and splenic surgery. (7) Previous radiation therapy to the left or right upper quadrant. (8) Previous gastric, hepatic, or splenic embolization. (9) Any history of portal venous hypertension. (10) Severe renal impairment resulting in unacceptable risk of contrast-induced nephropathy. (11) Pregnant or intend to become pregnant within one year. (12) History of severe bleeding disorder (platelet count less than 40,000). (13) Allergy to materials in the embolic agents. (14) Enrolled in another study. (15) Any patient who has a history of allergic reaction to iodinated contrast. (16) Abnormal baseline gastric emptying study. (17) Patients taking anti-coagulants (anti-platelets fine). (18) Patients currently taking or requiring chronic use of non-steroidal anti-inflammatory drugs (NSAID) or steroid medications. (19) Patients with any chronic upper gastrointestinal complaints such as pain, nausea or vomiting. (20) Patients with any history of peptic ulcer disease. (21) Patients with any indication of gastrointestinal bleeding as documented by positive stool guaiac and complete blood count with abnormalities. (22) Subjects with mesenteric atherosclerotic disease or abdominal angina should be excluded due to safety concerns. (23) Patients with known aortic disease, such as dissection or aneurysm. (24) Patients with comorbidities such as cancer. (25) Patients with any abnormality on their baseline esophagogastroduodenoscopy (EGD). (26) Patients with a CT Angiogram demonstrating an anatomical variant in gastric artery anatomy. (27) Patients with any contraindications for monitored anesthesia care or general surgery. (28) Patients with secondary causes of obesity such as Cushing's disease, hypothyroidism, or abnormal testosterone readings. (29) Patients with active substance abuse or alcoholism. (30) Patients with defined noncompliance with previous medical care. (31) Patients with certain psychiatric disorders such as schizophrenia, borderline personality disorder, uncontrolled depression, and mental/cognitive impairment that limits the individual's ability to understand the proposed therapy.

### REFERENCES

1. Lustig R. H., et al., Hypothalamic obesity caused by cranial insult in children: altered glucose and insulin dynamics and reversal by a somatostatin agonist, J Pediatr. 1999 August; 135 (2 Pt 1): 162-8.
2. Lustig R. H., et al., Octreotide therapy of pediatric hypothalamic obesity: a double-blind, placebo-controlled trial, J. Clin. Endocrinol. Metab. 2003 June; 88 (6): 2586-92.
3. Velasquez-Mieyer P. A., et al., Suppression of insulin secretion is associated with weight loss and altered macronutrient intake and preference in a subset of obese adults. Int. J. Obes. Relat. Metab. Disord, 2003 February; 27 (2):219-26.
4. Weiss C. R., et al., Clinical safety of bariatric arterial embolization: preliminary results of the BEAT obesity trial. Radiology. 2017; 283(2):598-608.
5. Bai Z. B., et al., Bariatric Embolization of the Left Gastric Arteries for the Treatment of Obesity: 9-Month Data in 5 Patients). Obes. Surg, 2018 April; 28(4):907-915.
6. Davies M. J., et al., Efficacy of Liraglutide for weight loss among patients with type 2 diabetes: The SCALE diabetes randomized clinical trial. JAMA. 2015; 314(7):687-99.
7. Buzga M, et al., Effects of the intragastric balloon MedSil on weight loss, fat tissue, lipid metabolism, and hormones involved in energy balance. Obes. Surg. 2014; 24(6):909-15.

What is claimed is:

1. A method for causing weight loss in a subject comprising introducing biodegradable microspheres into one or more of the subject's gastric arteries, wherein the microspheres (i) have a d₉₀ value from 40 μm to 500 μm; (ii) comprise polylactic acid (PLA) and/or polylactic co-glycolic acid (PLGA); (iii) carry a therapeutically effective amount of pharmaceutical octreotide; (iv) embolize gastric arterial vessels supplied by the one or more arteries into which they are introduced; and (v) release octreotide during embolization.

2. The method of claim 1, wherein the subject is human.

3. The method of claim 2, wherein the method comprises introducing the biodegradable microspheres into the subject's left gastric artery.

4. The method of claim 2, wherein the subject has a body mass index (BMI) of 40 or higher.

5. The method of claim 2, wherein each microsphere comprises PLA and PLGA.

6. The method of claim 5, wherein each microsphere comprises PLA and PLGA at a PLA:PLGA molar ratio from 40:60 to 50:50.

7. The method of claim 6, wherein each microsphere comprises PLA and PLGA at a PLA:PLGA molar ratio of 45:55.

8. The method of claim 2, wherein the biodegradable microspheres have a d₉₀ value from 50 μm to 200 μm.

9. The method of claim 8, wherein the biodegradable microspheres have a d₉₀ value from 70 μm to 150 μm.

10. The method of claim 2, wherein the therapeutically effective amount of pharmaceutical octreotide is from 5 mg to 50 mg.

11. The method of claim 10, wherein the therapeutically effective amount of pharmaceutical octreotide is from 10 mg to 20 mg.

12. The method of claim 10, wherein the therapeutically effective amount of pharmaceutical octreotide is from 20 mg to 30 mg.

13. The method of claim 2, wherein the embolization lasts from four to eight weeks.

14. A method for causing weight loss in a human subject having a BMI of 40 or higher comprising introducing biodegradable microspheres into the subject's left gastric artery, wherein the microspheres (i) have a d₉₀ value from 70 μm to 150 μm; (ii) comprise PLA and PLGA at a PLA:PLGA molar ratio of 45:55; (iii) carry from 10 mg to 20 mg of pharmaceutical octreotide; (iv) embolize gastric arterial vessels supplied by the left gastric artery; and (v) release octreotide during embolization.
